(19) United States
(12) Patent Application Publication
(10) Pub. No.: US 2012/0220945 A1
(43) Pub. Date: Aug. 30, 2012

(54) INFUSION KIT WITH A RETRACTABLE NEEDLE

(76) Inventors: Alberto Rizzo, Lucca (IT); Paquale Nardini, Villa Collemandina (LU) (IT); Armando Pasquinelli, Lucca (IT)

(21) Appl. No.: 13/505,856

(22) PCT Filed: Oct. 29, 2010
(86) PCT No.: PCT/IB10/54915

§ 371 Date: May 3, 2012

(30) Foreign Application Priority Data
Nov. 3, 2009 (IT) PR2009A000086

(51) Int. Cl. A61B 17/34

(52) U.S. Cl. 604/164.12

(57) ABSTRACT

Infusion kit includes: a butterfly needle having a stiff rod-shaped body, at least a pair of coplanar wings laterally and outwardly extending therefrom; a cavity for receiving a slide, supporting a sliding needle from which an element projects and engages a straight groove along the body; the element being longer than the cavity and projecting from the body, is insertable in a housing connected to the groove to compressibly block a spring between the slide and the cavity bottom wall; an infusion cannula including a central and hollow cylinder, a flexible element, at least a coupling having an inner passage; the flexible element and the passage communicating via the cavity to allow draining a fluid or the supply from the outside; the flexible element is hollow and allows insertion of the needle of the butterfly needle through the cavity; at least a pair of coplanar wings extend from the cylinder.

### FIELD OF THE INVENTION

The present invention relates to the field of the safety devices for medical instruments, and more particularly, it refers to an infusion kit having a retractable needle.

More specifically, the kit comprises a butterfly needle having a system for returning and protecting the needle and an infusion cannula, connected to the first element by an anti-rotational mechanical coupling.

### STATE OF THE ART OF THE BUTTERFLY NEEDLES

A butterfly needle is a steel needle having a intermediate length comprising plastic wings for fixing the needle to the skin. Moreover, it is provided with a rubber fitting adapted to be used for every kind of drug administration.

Currently, devices, mechanisms provided with different leverages or buttons for the protection of the needle or for activating the retention of the same are used.

A first group of needle protection devices is described, for example, in the patent document U.S. Pat. No. 6,719,737, in which the needle is coupled to a specific coating, such as protecting sheath, locatable and hinged to the end portion of the needle and adapted to snap on the same after it has been extracted.

A second group of protection systems are those that provide the activation of systems for retracting the needle, in a suitable receiving body, at the end of the injection.

Some examples of activation systems of the second group are: disclosed in documents U.S. Pat. No. 7,048,718, U.S. Pat. No. 5,549,571 and U.S. Pat. No. 5,779,679.

Referring particularly to the latter, it is to be observed that it provides the use of a double set of wings, two of these are used for introducing the needle in the patient's body and its stabilization, while the other two wings have each pawls for activating a needle holding cylinder so that, if they are folded, they relieve the needle from the main body and an inner spring returns the needle in the main body.

### STATE OF THE ART OF THE INFUSION CANNULA

A cannula is a flexible element made of rubber or other material, which is introduced in a body for promoting the drainage of the contents or for supplying it from the outside; in this case, the flexible element projects from a main stiff body provided with an inner passage connected to the supply connector.

### DISCLOSURE AND ADVANTAGES OF THE INVENTION

The object of the invention consists of providing a kit comprising, in combination, a butterfly needle and an infusion cannula integral to each other by a anti-rotational mechanical coupling, in order to have, in a first step, the possibility of inserting the cannula by the needle which is received in and projects from the cannula, and then the butterfly needle can be advantageously removed by keeping in position the cannula.

Another object of the invention consists of providing a system for retracting the butterfly needle, by activating said butterfly needle wings, directly after their folding. In this way, the operator does not directly act on the activating device for returning the needle but, in an easier way, it acts on the side wings, which, in turn, compress said activating device causing the return of the needle. The advantage of said second object consists of avoiding inadvertent injections due to the use and handling of butterfly needles after the injection.

### BRIEF DESCRIPTION OF THE FIGURES

These and other characteristics will be better understood by the following description of some embodiments shown in an illustrative and not limiting way in the following drawings:

FIG. 1 shows a perspective view of the infusion kit of the invention,

FIG. 2 shows the cannula of the infusion kit,

FIG. 3 shows only the butterfly needle of the infusion kit, the latter can be used alone or in combination with the cannula of FIG. 2,

FIG. 4 shows three plan views of the kit of FIG. 1,

FIG. 5 shows a cross-section view of the infusion kit of the invention.

### DESCRIPTION OF THE INVENTION

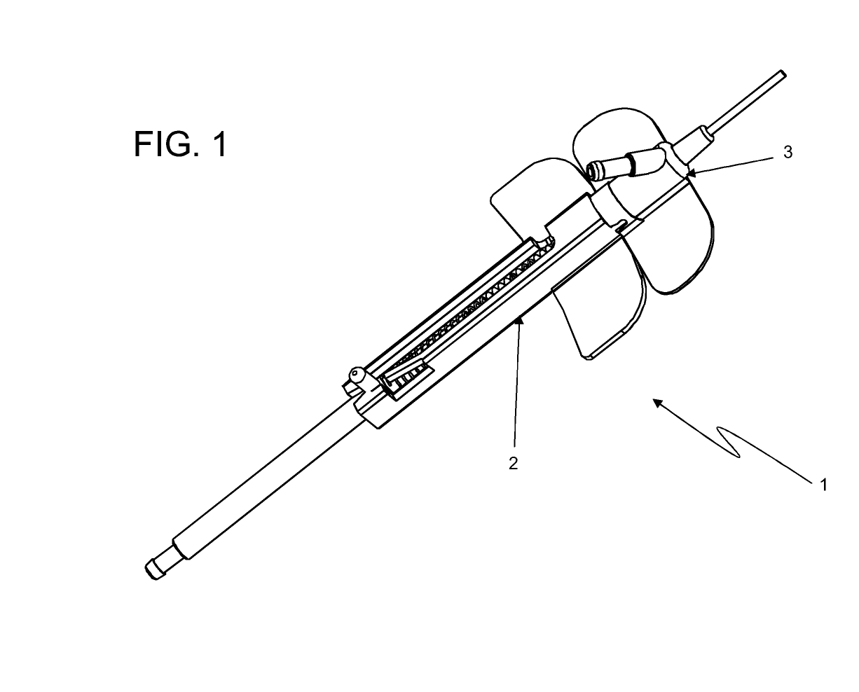

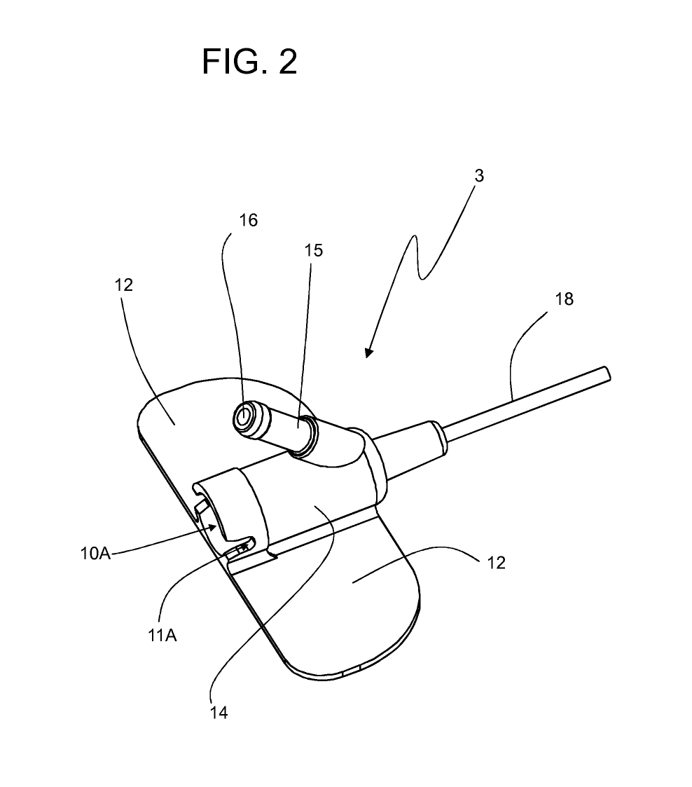

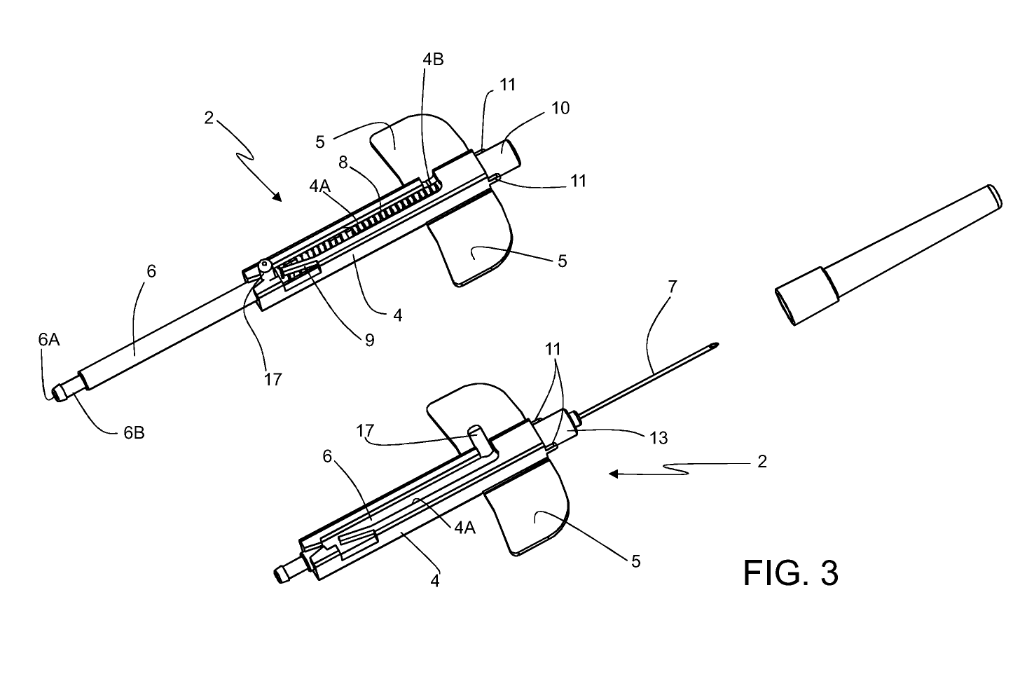

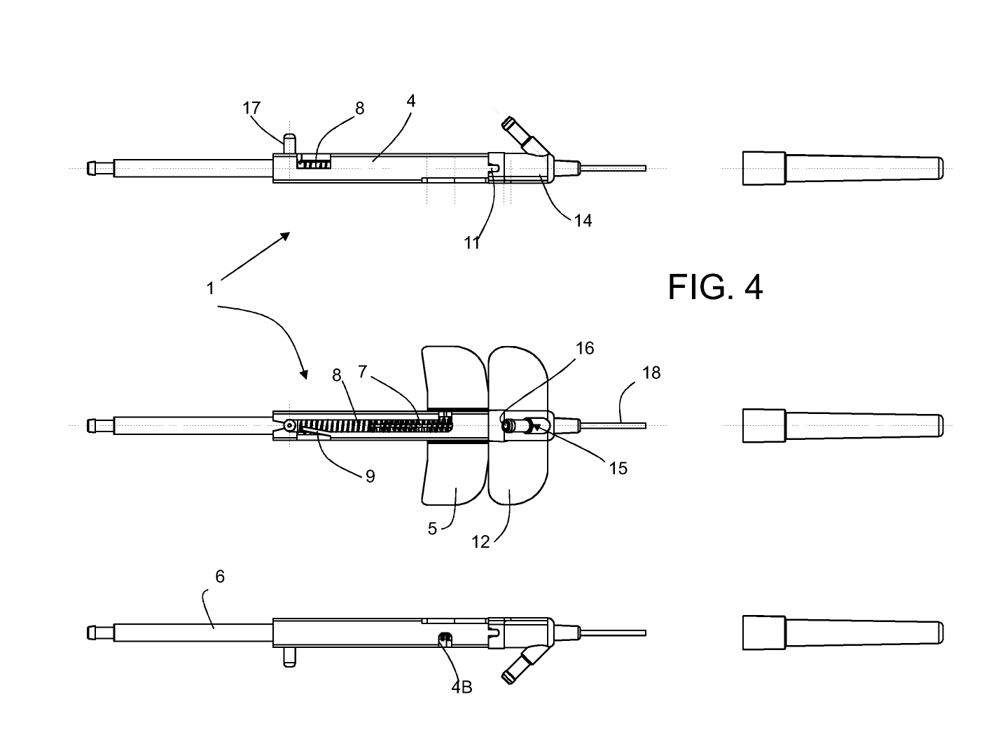

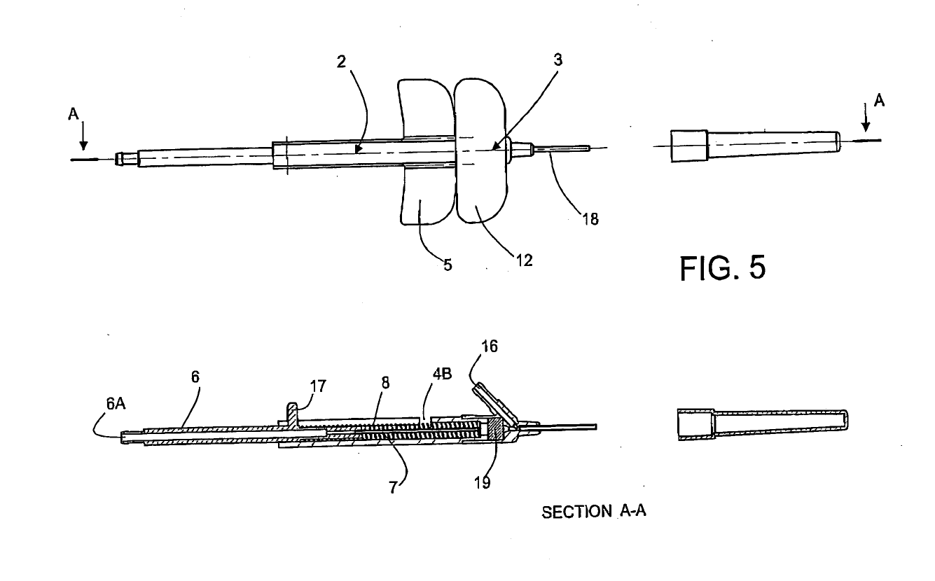

Referring particularly to FIGS. 1, 2, 3, 4 and 5, 1 generally shows an infusion kit comprising a butterfly needle 2 coupled and coupable to an infusion cannula 3. Both the needle 2 and the cannula 3 comprise at least one pair of coplanar wings 5 and 12, laterally projecting from the respective components and having a large flat surface, whose function consists of stabilizing the corresponding component after the introduction in the patient. Once the butterfly needle 2 and the cannula 3 have been joined, the wings 5 and 12 are coplanar.

The needle 2 and the cannula 3 are interconnected by mechanical and anti-rotational means 10, 10A and 11, 11A, that is by a lock system of the male/female type.

According to the this shown embodiment, the interconnecting system is coaxial with the needle 2 and cannula 3 and is formed by a cylindrical hub 10 projecting from the needle 2 suitable to be inserted in a corresponding female cavity 10A of the cannula 3; at least a linear tab 11 laterally projects from said hub 10 and it is adapted to be inserted in a corresponding slot 11A of the cannula. In the example, there are two opposed tabs 11 preferably located on the midplane of the hub.

The above mentioned embodiment is a possibility of implementing the mechanical and anti-rotational interconnection between the retractable needle and the cannula; other variants of the embodiment are provided without departing from the scope of the invention. For example, a second embodiment can provide a hub and the corresponding cavity, having a quadrangular cross-section, instead of a circular cross-section, so that the same coupling can act as a lock and anti-rotational system. A third variant can provide a pair of spaced pins, projecting from the butterfly needle, adapted to be inserted in at least two corresponding cavities of the cannula 3 body in order to act as a simultaneous lock and anti-rotational system, instead of the hub 10. The butterfly needle 2 and the cannula 3 will be now described specifically.

The butterfly needle 2 comprises a substantially rod-shaped body stiff 4 having a quadrangular cross-section, from which two wings 5 laterally extend, having the function of stabilizing the component on the patient and of activating the return of the needle as it will be explained in the following.

Along the axis AA of the rod-shaped body there is a cavity 4A adapted to receive a slide 6 having a corresponding size; an element 17 projecting from the cursor 6 engages a longitudinal opening 4A, made along the axis AA; said element 17 is longer than the opening 4A so that projects from the body 4.

The slide 6 supports a metal needle 7; said needle 7 is coaxial with the slide 6 and therefore with the body 4 once said components are inserted in the corresponding cavity.

Inside the slide 6 there is a passage 6A, coaxial with the axis AA, which allows the flow of a fluid from the needle 7 at the opposed end. The needle 7 is also pierced so that the fluid can exit or can be drawn.

The component 2 further comprises a spring 8 located in the body 4 cavity and specifically between the slide 6 and a bottom wall 4C of the cavity. Practically, the spring 8 works in a compression state when the slide 6 is inserted in the cavity and the corresponding needle 7 projects from the body 4, passing inside the spring 8 and through a suitable passage hole made in said hub 10. Said operative arrangement (the slide 6 compressing the spring 8 and the needle projecting from the body 4) is kept by keeping blocked in position the slide 6, due to its element 17 which is positioned in a housing 4B connected to the groove 4A; the element 7 and the slide 6 are rotated so that the introduction in said housing 4B makes useless the thrust of the spring 8, which is now compressed between the slide 6 and the bottom wall 4C.

The housing 4B is made in correspondence with the wings 5, that is, so that the element 7 will interact with said wings 5. What has been discussed is the triggering action for returning the needle 7; actually, the infusion kit 1 comprises a system for protecting and returning the needle which acts after the folding of the wing 5 serving as a stabilizing means for the butterfly needle 2.

More specifically, the term “folding of the wings 5” should be interpreted as the action of closing the same towards the body 4, from the side of the groove 4A. During said folding action, the wing 5 acts against the element 7 unseating the latter from its seat or housing 4B and returning it in the groove 4A: at this point, the spring 8, since it does not have mechanical obstacles to its action, snaps and retracts the slide 6 and needle 7; the length of the body 4 cavity is sufficient for completely containing the needle 7 which remains protected at its inside.

Possibly, on the body 4 there is also a safety device which avoids a further sliding in an opposite way; with reference to the figures, the device is for example formed by an extension 9 of the flexible body 4, which unidirectionally enters in the groove 4A, in this way prevents the reactivation, or anyway also a slight sliding, of the element 7.

The cannula 3 comprises:
a hollow central cylinder 14, in the example its cavity is the same cavity 10A shown for the interconnection with the butterfly needle 2,
a flexible hollow element 18 made of rubber or other material, which is introduced in said cylinder 14 and projects frontally from the latter;
at least a coupling 15 having an inner passage 16,
at least a pair of coplanar wings 12 outwardly extending from the cylinder 14.

The flexible element 18 and the passage 16 communicate through the cylinder 14 cavity in order to allow the drainage of a liquid or its supply from the outside. The flexible element 18 is hollow and said cavity has a size such as to allow the passage of the needle 7, which occurs after the attachment to the component 2 by the above described interconnecting means.

For avoiding dispersions of fluid inside the body 4 cavity, the infusion kit 1 comprises a fluid tight element 19 located inside the cylinder 14 cavity 10A; once the needle 7 has been returned inside the body 4, the element 17 seals the passage so that it permits a fluid exchange only between the flexible element 18 and the passage 16.

1. Infusion kit (1) characterized by the fact it in combination comprises:
a. a butterfly needle (2) formed by a rod-shaped stiff body (4), at least a pair of coplanar wings (5) laterally and outwardly extending from the butterfly needle; a cavity (4A) along the axis (AA) of the body (4) is adapted to receive a slide (6) supporting a sliding needle (7) from which an element (17) projects and engages a straight groove (4A) made along the body (4); said element (17), longer than the opening (4A) in order to project from the body (4), can be inserted in a housing (4B) connected to the groove (4A) in order to compressibly block a spring (8), between the slide (6) and the bottom wall (4C) of the body (4) cavity;
b. an infusion cannula (3) comprising a central and hollow cylinder (14), a flexible element (18) made of rubber or other material, at least one coupling (15) having an inner passage (16); the flexible element (18) and passage (16) communicate through the cylinder (14) cavity in order to permit the drainage or supply of the fluid from the outside; said flexible element (18) is hollow and the needle (7) of the butterfly needle (2) can be inserted through said cavity; at least one pair of coplanar wings (12) extend from said cylinder (14), said needle (2) and said cannula (3) being joinable by anti-rotational mechanical coupling means (10, 10A, 11, 11A), in order to have, in a first step, the possibility of inserting the cannula by the needle which is inside and projecting from the same, and then of removing the butterfly needle by keeping in position the cannula.

2. Kit, according to claim 1, characterized by the fact said anti-rotational mechanical coupling is formed by a hub (10) having a cylindrical cross-section, projecting from the needle (2) adapted to be inserted in a corresponding female cavity (10A) of the cannula (3); at least one tab (11) laterally projects from said hub (10) and is adapted to be inserted in a corresponding slot (11A) of the cannula.

3. Kit according to claim 1, characterized by the fact said anti-rotational mechanical coupling comprises a quadrangular cross-section hub project from the needle (2) to enter a corresponding quadrangular cross-section cavity (10A) and the coupling acts as an anti-rotational lock system.

4. Kit according to claim 1, characterized by the fact said anti-rotational mechanical coupling comprises at least one pair of spaced pins projecting from the butterfly needle, adapted to be inserted in at least two corresponding cavities of the cannula (3) body, in order to act simultaneously as a lock and anti-rotational system.

5. Kit according to claim 1, characterized by the fact said housing (4B) is located in a corresponding operative range of the wings (5), so that the element (7), associated to said housing, interacts with said wings (5) after the folding of the same.

6. A butterfly needle (2) comprising a stiff rod-shaped body (4), at least one pair of coplanar wings (5) laterally and outwardly extending from the body; a cavity (4A) along the axis (AA) of the body (4), is adapted to receive a slide (6) supporting a sliding needle (7) from which an element (17) extends and engages a straight groove (4A) made along the body (4); said element (17), longer than the opening (4A) in order to project from the body (4), can be inserted in a housing (4B) connected to the groove (4A) in order to compressibly block a spring (8), between the slide (6) and the bottom wall (4C) of the body (4) cavity, characterized by the fact it comprises anti-rotational mechanical coupling means (10, 11) for housing a cannula or an infusion device.
